(19) United States
(12) Patent Application Publication
(10) Pub. No.: US 2013/0282057 A1
(43) Pub. Date: Oct. 24, 2013

(54) Skin Closure Clamping Device And Procedure

(71) Applicants: Sheila Green, Dillsburg, PA (US); R. Michael Johnson, York, PA (US); Pietro Ranieri, Haddonfield, NJ (US); Kristin Chrouser, St. Louis Park, MN (US)

(72) Inventors: Sheila Green, Dillsburg, PA (US); R. Michael Johnson, York, PA (US); Pietro Ranieri, Haddonfield, NJ (US); Kristin Chrouser, St. Louis Park, MN (US)

(73) Assignee: JHPIEGO CORPORATION, Baltimore, MD (US)

(21) Appl. No.: 13/911,479

(22) Filed: Jun. 6, 2013

Related U.S. Application Data
(63) Continuation-in-part of application No. 13/114,692, filed on May 24, 2011.

(51) Int. Cl. A61B 17/064

(52) U.S. Cl. CPC A61B 17/064
USPC 606/220

(57) ABSTRACT

A skin closure clamping device includes: (a) a strip whose outer surface includes a front portion that has a number of cavities therein, (b) an assortment of pegs or male staples fabricated from a biodegradable material, each having distal and proximal ends, and wherein each of the pegs' distal ends is configured to pierce a patient's skin, and each of the pegs' proximal ends is configured to allow it to be detachably fitted into one of the cavities of the strip, and each peg's outer surface proximate its distal end having a ratcheting mechanism, and (c) a female member having an outer surface with a front portion that includes an opening configured to receive the pegs' distal ends in such a manner that the pegs' ratcheting mechanisms interact with the opening to adjustable lock together each of the pegs to the female member so as to apply a compressive force between them that aids in holding a patient's skin together while it heals.

### CROSS-REFERENCE TO RELATED APPLICATION

This application is a continuation-in-part application of and claims the benefit of U.S. patent application Ser. No. 13/114,692, filed May 24, 2011 by the present inventors and entitled “Circumcision Device And Procedure.” The teachings of this application are incorporated herein by reference to the extent that they do not conflict with the teaching herein.

### BACKGROUND OF THE INVENTION

1. Field of the Invention

The present invention relates generally to surgery devices and methods. More particularly, the present invention relates to a skin closure device and the procedure for its use.

2. Description of the Related Art

Wound closure devices such as sutures, staples and tacks have been widely used in superficial and deep surgical procedures in humans and animals for closing wounds, repairing traumatic injuries or defects, joining tissues together (bringing severed tissues into approximation, closing an anatomical space, affixing overlapped tissue layers together, creating an anastomosis between two hollow/luminal structures, adjoining tissues, attaching or reattaching tissues to their proper anatomical location), attaching foreign elements to tissues (affixing medical implants, devices, prostheses and other functional or supportive devices), and for repositioning tissues to new anatomical locations (repairs, tissue elevations, tissue grafting and related procedures) to name but a few examples.

Sutures are often used in these applications. Sutures typically consist of a filamentous suture thread attached to a needle with a sharp point. Suture threads can be made from a wide variety of materials including bioabsorbable (i.e., that break down completely in the body over time durations of 90 days or longer) or biodegradable, or non-absorbable (permanent; non-degradable) materials. Absorbable sutures have been found to be particularly useful in situations where suture removal might jeopardize the repair or where the natural healing process renders the support provided by the suture material unnecessary after wound healing has been completed; as in, for example, completing an uncomplicated skin closure. To use an ordinary suture, the suture needle is advanced through the desired tissue on one side of the wound and then through the adjacent side of the wound. The suture is then formed into a “loop” which is completed by tying a knot in the suture to hold the wound closed.

Non-ordinary sutures include those which are commonly referred to as knotless or self-retaining sutures. These differ from ordinary sutures in that their threads are usually larger in diameter and possess numerous barbs on the thread which anchor such self-retaining sutures into the tissue following deployment and resist movement of the suture in a direction opposite to that in which the barbs face, thereby eliminating the need to tie knots to affix adjacent tissues together. See, for example, U.S. Pat. Nos. 5,374,268, 5,584,859, 5,931,855, 6,241,747, 6,264,675 and 6,848,152.

Many varieties of staples or surgical fasteners are known in the art. A conventional staple generally includes a pair of legs having tips that are adapted to penetrate tissue and wherein the bases of these legs are connected by a backspan or longitudinal element from which the legs extend. See, for example, U.S. Pat. Nos. 7,794,475, 6,551,333 and 6,747,121.

A somewhat less common staple is the multi-part staple device. In addition to a pair of legs with a backspan and tissue penetrating tips, a multi-part staple includes a retainer piece or female member which has openings through which the legs' tips pass before being bent over and further locked into the female member. Such fasteners are often not intended to be unlocked or removable, and are generally made of a absorbable or degradable material. See, for example, U.S. Pat. Nos. 8,348,972, 6,273,897, 4,627,437 and 4,532,927.

In general, a surgical fastening apparatus is used to apply such staples to the edges of a wound. For example, such a fastening apparatus may include a head having opposed jaws that contain carefully aligned staples in one jaw (i.e., cartridge) and comparably aligned retainers in the other jaw (i.e., anvil). During a surgical procedure, the fastening apparatus is used to grip or clamp a patient's tissue between the apparatus' jaws such that individual staples can be ejected from the cartridge so that the staples' pointed tips penetrate the gripped tissue and are driven into apertures on the retainers.

Despite the widespread use of such fasteners and fastening apparatus, there are many emerging nations where such tools have limited usefulness because large numbers of people in these areas don't have access to modern operating rooms staffed by fully qualified medical practitioners. In these nations, there is often the need for an improved means of performing skin closure procedures in situations where fully qualified medical practitioners and facilities are not available.

An objective of the present invention is to provide such means. A version of the skin closure device of the present invention has been designed for use by caregivers who can be relatively easily taught how to use it and to then perform the novel skin closure procedures of the present invention.

Additionally, there exists the opportunity to improve upon staples of the type that are intended to be used in more standard applications (e.g., dermal, neurology, gynecological and laparoscopic suturing situations). The areas in which improvements are always being sought for such staples include: (a) decreasing surgical times by configuring staples so that they can be used to more quickly close a wound, and (b) controlling and minimizing the excessive stresses that may be imposed upon and damage the tissue adjoining the edges of an inserted staple. Another objective of the present invention is to provide an improved staple that offers improvements in both these areas.

### SUMMARY OF THE INVENTION

Recognizing the need in emerging nations for the development of improved skin closure methods and devices that can be used in situations where fully qualified medical practitioners and facilities are often not readily available, the present invention is generally directed to satisfying this need.

In accordance with a preferred embodiment of the present invention, a skin closure clamping device which reasonably skilled and trained caregivers can use to close a skin wound painlessly, safely, effectively, quickly and for a minimal cost includes: (a) a strip whose outer surface includes a front portion that has a number of cavities therein, (b) an assortment of pegs or male staples fabricated from a degradable material, each having distal and proximal ends, and wherein each of the pegs' distal ends is configured to pierce a patient's skin, and each of the pegs' proximal ends is configured to allow it to be detachably fitted into one of the cavities of the strip, and each peg's outer surface proximate its distal end having a ratcheting mechanism, and the peg's outer surface proximate its proximal end having a recess which is configured to increase the peg's surface area, and (c) a female member having an outer surface with a front portions that includes an opening which is adapted to receive the pegs' distal ends in such a manner that the pegs' ratcheting mechanisms interact with the opening to adjustable lock together each of the pegs to the female member so as to apply a compressive force between them that aids in holding a patient's skin together while it heals.

Alternatively, the present invention may be considered to be the use of the clamping device described above to allow reasonably skilled and trained caregivers to close a skin wound painlessly, safely, effectively, quickly and for a minimal cost.

Additionally, the present invention may be considered to be the process or method by which a reasonably skilled and trained caregiver can use the clamping device described above to close a skin wound painlessly, safely, effectively, quickly and for a minimal cost.

Thus, there has been summarized above (rather broadly and understanding that there are other preferred embodiments which have not been summarized above) the present invention in order that the detailed description that follows may be better understood and appreciated.

### BRIEF DESCRIPTION OF THE DRAWINGS

FIG. 1(a) is a top view of a preferred embodiment of the present invention.

FIG. 1(b) is a side view of a preferred embodiment of the present invention.

FIG. 1(c) is a rear view of a preferred embodiment of the present invention.

FIG. 2 is a perspective view of a preferred embodiment of the strip element of the present invention.

FIG. 3 is a perspective view of a first preferred embodiment of the peg element of the present invention.

FIG. 4 is a perspective view of a preferred embodiment of the female member of the present invention.

FIGS. 5-8 show side views of alternative designs for the outer surface of the present invention's peg or male staple.

FIGS. 9-10 show perspective and cross-sectional views, respectively, of an alternative design for the strip and female members of the present invention in which a single peg is attached to each strip.

### DETAILED DESCRIPTION OF THE INVENTION

Before explaining at least one embodiment of the present invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

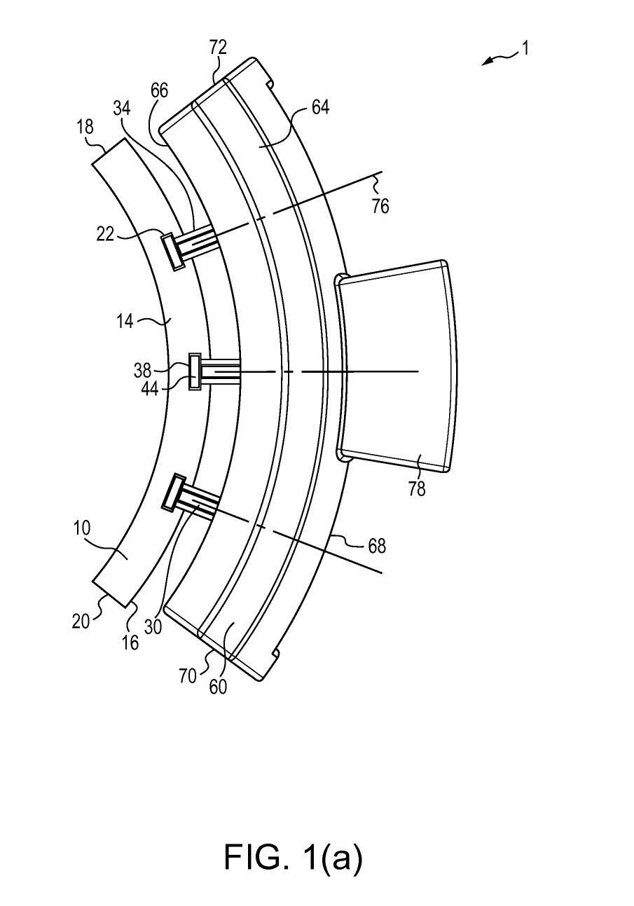

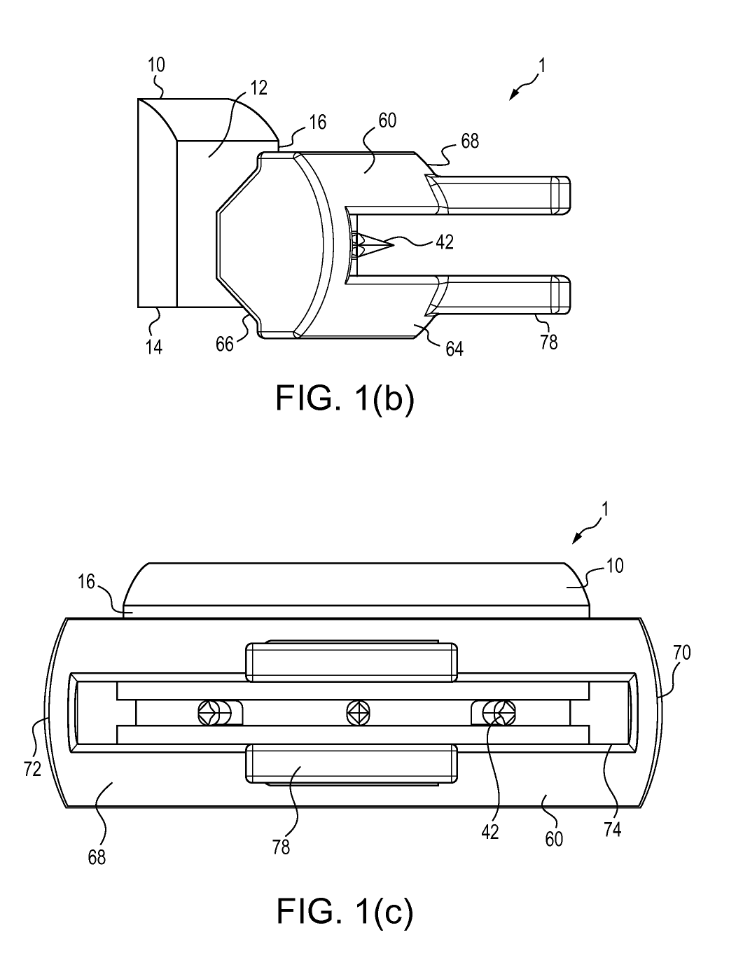

The current invention includes both a skin closure clamping device and the skin closure method or procedure that is made possible by this device. FIGS. 1(a)-1(c) show respective top, side and rear views of a preferred embodiment of the present invention. It is seen to consist of a clamping device 1 that includes a strip or cartridge 10 that acts as an alignment device for a number of degradable pegs or axial members or male staples 30 which each have a pointed end or a configuration 42 that is adapted to pierce a patient's skin, on the staple or peg's distal end 36.

The staple's distal end 36 passes through the opposing edges 3 on either side of a wound and then adjustably and detachably locks via a ratchet 46 with an opening 74 in the front portion 66 of a retainer piece or female member 60 to compress and seal the wound's edges between this retainer member's front portion 66 and either a strip's front portion 16 or, if no cartridge or strip is attached to a staple's base, the staple's base 44 itself. This design allows for the staple 30 to replace the function of sutures in suitably chosen wound closing applications. Eliminating sutures with the staple creates a new, simpler type of operation that caretakers or caregivers without an extensive education can perform. The caretakers can even manually apply some of these clamping apparatus with especially designed cartridges 10 and female members 60 and without the need of additional staplers, tools or fastening apparatus.

Additionally, the staples 30 in this clamping device 1 are preferably fabricated from a degradable material so that they do not require surgical removal. As the staple begins to degrade it will lose its holding ability and the staple will disengage from the female member 60 and fall off of the patient.

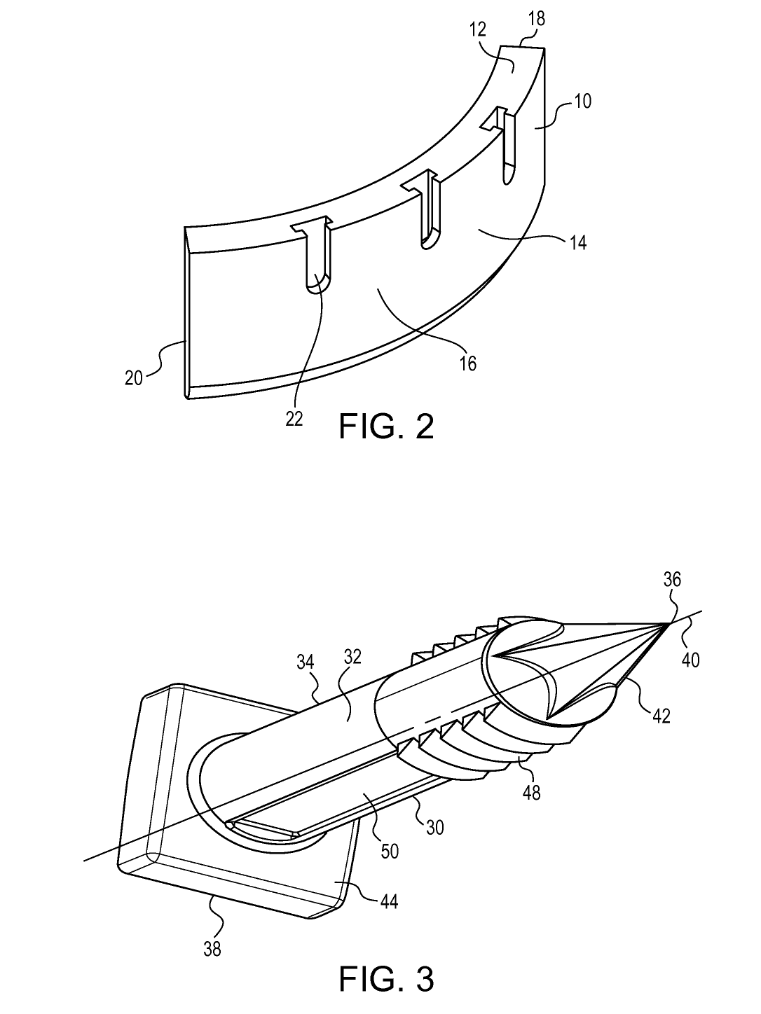

Shown in FIG. 2 is a perspective view of a preferred embodiment of the strip 10 element of the present invention. It is seen to have an interior region 12 that has an elongated outer surface 14 that includes a front portion 16 that extends between the distal 18 and proximal 20 ends of the strip. Its' front portion has a number of cavities 22 that are distributed between the strip's ends and configured such that each of these cavities extends into the interior region of the strip and can accommodate the proximal 38 end of a staple 30.

It can also be noted that the combined surface area of the openings of these cavities is only a fraction of the total surface area of the strip's front portion 16—alternatively, the combined cross-sectional surface area of the staples is only a fraction of the total surface area of the strip's front portion 16. This design feature proves useful when the strip 10 is used to apply a more uniform compressive force to a patient's adjoining skin than could be applied if only staples or pegs 30 alone were to be used to try to compress and hold together the patient's skin on the adjoining edges of a wound.

Shown in FIG. 3 is a side view of a preferred embodiment of the staple 30 element of the present invention. It is seen to have an interior region 32 with an elongated outer surface 34 that has distal 36 and proximal 38 ends. Between these ends extends an axial centerline 40 that serves to define the axial length, L, between the staple's ends.

The distal end of this staple has a pointed end or barb 42 or a configuration that is adapted to pierce the skin of a patient and its proximal end has a base 44 configuration that is adapted to allow it to be detachably fitted into one of the strip's cavities 22. This base 44 configuration also serves to set the intersection angle that the staple's centerline axis 40 makes with the strip's front portion 16.

Near a staple's distal end 36 are a number of spaced-apart ratchets 46 or ratcheting mechanisms 48 (e.g., the staple's outer surface has a saw-tooth shape) that are configured to extend over a specified distance of a staple's axial length, L. This length serves to set the adjustability for the actual, desired, in-use, axial length that is available to set the thickness of the tissue which is to be joined between the strip's and the female member's front portions when they have been locked together to hold and compress the opposing edges of a wound.

Meanwhile, near a staple's proximal end 38 is a recess or cavity 50 that extends into the staple's interior region and is configured to increase the surface area of the staple or to vary the total surface area of the staple's outer surface per its unit volume. This is done as a means of helping to control the time that it will take for the staple to sufficiently degrade that it loses its holding ability and disengages from the female member 60 and falls off of the patient.

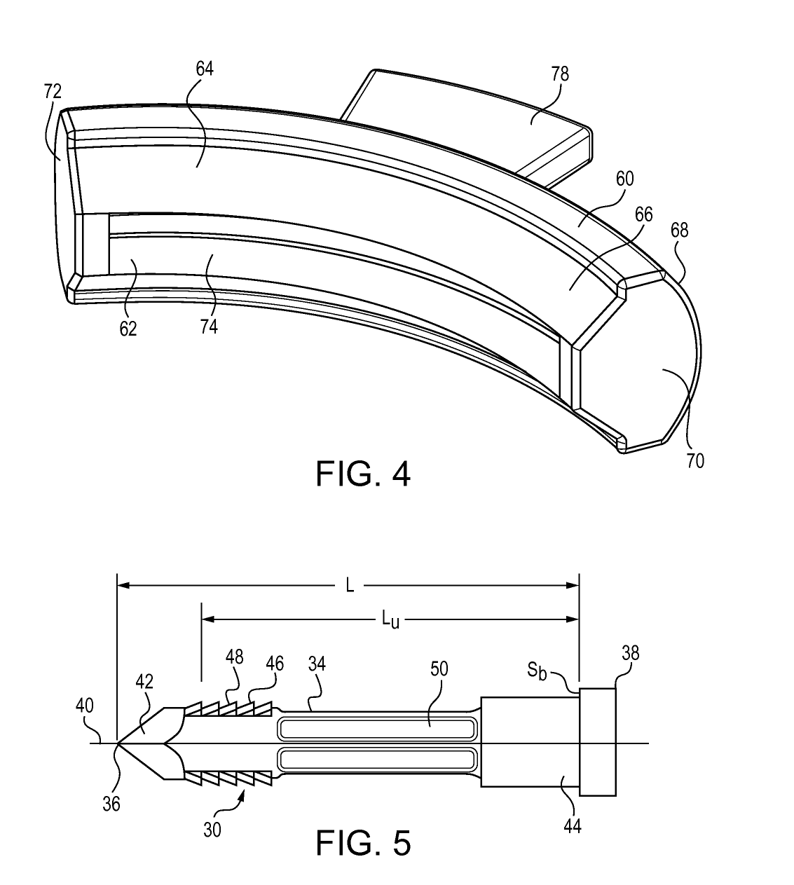

FIG. 4 is a perspective view of a preferred embodiment of the female member 60 of the present invention. It is seen to have an interior region 62, an elongated outer surface 64 that includes front 66 and rear 68 portions that extend between its distal 70 and proximal 72 ends. The front portion has one or more openings 74 that are each configured with a centerline 76 that allows them to align with and receive the staple's distal ends 36 and to adjustably lock with the staple's ratchets so that the axial length between the female member front 66 portion and a staple's base 44 is adjustably set to a desired axial length to yield optimum compression of the pierced tissue held between these opposing surfaces 66, 44.

Since this member is intended to be applied or attached to staples manually and without the aid of fastening apparatus or devices and in certain situations by a relatively untrained caregiver, it has attached to its rear portion a grasping member 78 which has a configuration that allows it to be grasped by the hand of a caregiver and used to help align this member's opening with a staple's axial centerline 40. The staple's base can also be similarly configured so the whole device can be easily grasped in the hands of a caregiver and applied manually, without the aid of a fastening apparatus.

It can also be noted that the surface area of the female member's front portion (i.e. a first specified surface area), exclusive of the area of its opening, is approximately equivalent to that of the surface are of the strip's front portion (i.e. a second specified surface area). This ratio is maintained in order to allow a relatively minimal, uniform compressive force to be applied around the edges of a wound that is to be closed by the clamping device.

After much experimentation, it was found that suitable materials from which to fabricate the present invention's degradable staple 30 is a combination of L-lactide and glycolide polymers in a ratio range of 1/10 to 1/6, with a preferred ratio of approximately 1/9. This preferred combination is generally equivalent to PURASORB® PLG 1017, which is manufactured by Purac Biomaterials, Gorinchem, The Netherlands. Once molded, the staple is subjected to varying degrees of gamma radiation and other environmental factors in order to condition it such that it possesses the property that it will degrade when placed in a patient's skin during the desired period of six to twenty days. The strip 10 and the female member 60 of the present invention are made from the medical grade of either a polypropylene, polyethylene, or nylon material.

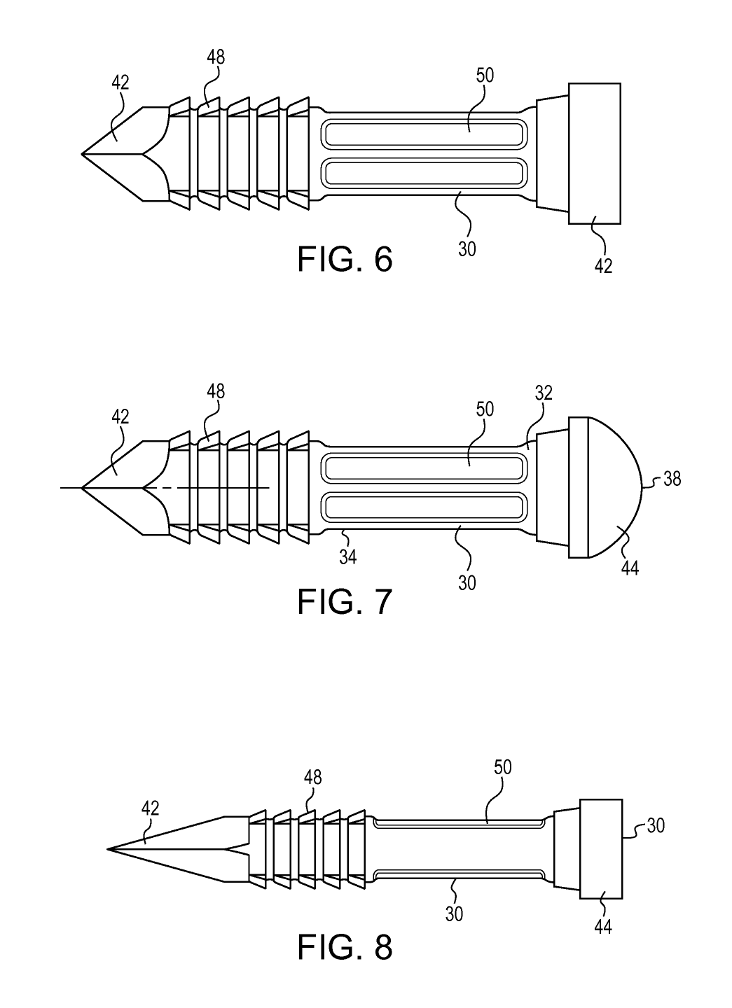

Some of the many alternative designs for the outer surface 34 of the present invention's peg or male staple are shown in FIGS. 5-8. These are all seen to have the characteristic pointed, piercing or barbed distal 36 ends and the bases 44 that are configured to fit into and mate with the cavities 22 in the front portion of the strips 10 that are used to align and house the staples 30.

A key feature of this improved wound closing device is the adjustability in the magnitude of the compression forces that can be applied to a wound's edges. This feature is achieved by fabricating these staples 30 so that they have a number of ratchets 46 or ratcheting mechanisms 48 spread along the staple's axial length, L. These ratchets 46 give the whole wound closing procedure adjustability—a feature that other stapling methods do not have.

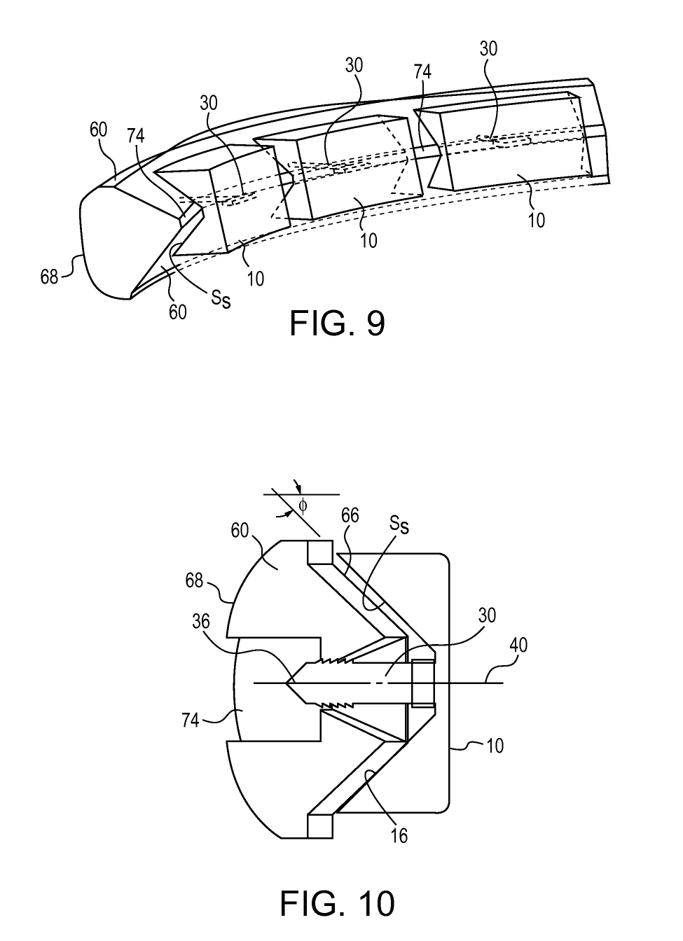

This adjustability comes about in a number of ways. First, the in-use axial length, Lᵤ, which is defined as the length between a staple's base and the specific ratchet member 46 to which the female member attaches, can be adjustably selected as appropriate by a care giver to lock the edges of the opening 74 in the front portion 66 of the retainer piece or female member 60 to the staple. Second, the surface area, S_{b}, of the staple's base 44, or if this base is attached to a strip's front portion 16, the surface area, Sₛ, of the strip's front portion 16, can be sized, consistent with any required spacing between the staples in order to adequately close a wound, in such a manner so as to uniformly distribute and minimize the compressive forces that are placed upon the tissue between such a multi-part staple's adjoining faces 66, 44, 16. See FIGS. 9-10. Note also how the base 44 of the staple, or a strip's front portion 16, can be oriented at an angle Ø with respect to the staple's centerline axis 40 so as to further increase the surface area of the tissue that is being uniformly compressed so as to minimize the stresses that are applied to the tissue that is being joined.

There are many alternative designs for the strip and female members of the present invention. One of these is shown in the respective perspective and side views of FIGS. 9 and 10. In this instance, the female member or female staple holder is a portion of a circular ring-shaped structure having a convex front portion 66 and a rounded rear portion 68. Designed into this staple holder is a trough-like opening 74 which extends between the female member's front 66 and rear 68 portions and is sized to accept and adjustably fit with the distal 36 ends of degradable staples 30 that are each individually attached to a strip or cartridge 10 whose front portion 16 has complimentary concave curvature so as to allow it to align with and adjoin the female member's front 66 portion for applying a uniform compressive force between these two opposing surfaces.

Since numerous modifications and changes will readily occur to those skilled in the art, it is not desired to limit the invention to the exact construction and operation shown and described herein. Accordingly, all suitable modifications and equivalents may be resorted to, falling within the scope of the invention that is hereinafter set forth in the claims to the invention.

We claim:

1. An improved skin closure device that a caregiver uses for joining tissues together, said device of the type having a staple with an elongated outer surface that extends between a distal end which is configured to pierce the skin of a patient and a proximal end, and an axial centerline that serves to define the axial length between said staple ends, a female member having an outer surface that includes a front portion with an opening which is configured to align with said staple axial centerline to receive said staple distal end, wherein the improvements to said skin closure device comprising:
a base affixed to the proximal end of said staple and said base having a configuration that is adapted to provide a specific surface area for contact with said female member front portion, and wherein said base specific surface area is chosen so as to minimize the stresses that are applied to said tissue that is being joined,
a ratchet mechanism affixed to the outer surface of said staple and said ratchet mechanism having a configuration that is adapted to interact with and adjustably lock said staple distal end to said female member opening so that the axial length between said female member front portion and said staple base is adjustably set to a desired in-use, axial length which is commensurate with the thickness of the tissue which is to be joined,
an orientation, with respect to said staple axial centerline, for said staple base that is characterizible in terms of an angle, Ø, which is chosen so as to minimize the stresses that are applied to said tissue that is being joined, and a biodegradable material from which said staple is fabricated.

2. The improved skin closure device as recited in claim 1, wherein:
said ratchet mechanism configuration further adapted to detachably lock said staple distal end to said female member opening.

3. The improved skin closure device as recited in claim 1, wherein:
said staple outer surface having a configuration that is adapted to have a total surface area per unit volume of said staple that is set so as to help control the duration of time required for an implanted staple to biodegrade in a patient.

4. The improved skin closure device as recited in claim 2 wherein:
said staple outer surface having a configuration that is adapted to have a total surface area per unit volume of said staple that is set so as to help control the duration of time required for an implanted staple to biodegrade in a patient.

5. The improved skin closure device as recited in claim 1, wherein:
said staple base and female member each having a configuration adapted to enable said device to be grasped in the hands of a caregiver and applied manually, without the aid of a fastening apparatus, to the edges of said tissue that is to be joined.

6. The improved skin closure device as recited in claim 2, wherein:
said staple base and female member each having a configuration adapted to enable said device to be grasped in the hands of a caregiver and applied manually, without the aid of a fastening apparatus, to the edges of said tissue that is to be joined.

7. The improved skin closure device as recited in claim 3, wherein:
said staple base and female member each having a configuration adapted to enable said device to be grasped in the hands of a caregiver and applied manually, without the aid of a fastening apparatus, to the edges of said tissue that is to be joined.

8. The improved skin closure device as recited in claim 4, wherein:
said staple base and female member each having a configuration adapted to enable said device to be grasped in the hands of a caregiver and applied manually, without the aid of a fastening apparatus, to the edges of said tissue that is to be joined.

9. The improved skin closure device as recited in claim 1, wherein:
said biodegradable material is a combination of L-lactide and glycolide polymers, with the ratio of said polymers being in the range of 1/10 to 1/6.

10. The improved skin closure device as recited in claim 2, wherein:
said biodegradable material is a combination of L-lactide and glycolide polymers, with the ratio of said polymers being in the range of 1/10 to 1/6.

11. The improved skin closure device as recited in claim 4, wherein: said biodegradable material is a combination of L-lactide and glycolide polymers, with the ratio of said polymers being in the range of 1/10 to 1/6.

12. The improved skin closure device as recited in claim 8, wherein:
said biodegradable material is a combination of L-lactide and glycolide polymers, with the ratio of said polymers being in the range of 1/10 to 1/6.

13. The improved skin closure device as recited in claim 9, wherein:
said ratio of L-lactide and glycolide polymers has a preferred value of approximately 1/9.

14. The improved skin closure device as recited in claim 12, wherein:
said staple is fabricated by subjecting said staple to varying degrees of environmental factors in order to condition said staple such that said staple possesses the property that said staple biodegrades in a duration of between six to twenty days.

15. The improved skin closure device as recited in claim 1, wherein:
said female member configuration further adapted to include a rear portion that has a grasping member which has a configuration that is adapted to allow said grasping member to be grasped by a caregiver and used to help align said opening of said female member with said axial centerline of said staple.

16. The improved skin closure device as recited in claim 1, wherein the improvements to said skin closure device further comprising:
a strip having an outer surface that includes a front portion with a cavity therein, and said cavity having a configuration adapted to accommodate said staple base and to detachably hold said staple in such a manner so as to aid in aligning the axial centerline of said staple with the opening of said female member during the adjustable locking of said staple distal end to said female member.

17. An improved skin closure method that a caregiver uses for joining tissues together, said method of the type comprising the steps of utilizing a staple with an elongated outer surface that extends between a distal end which is configured to pierce the skin of a patient and a proximal end, and an axial centerline that serves to define the axial length between said staple ends, utilizing a female member having an outer surface that includes a front portion with an opening which is configured to align with said staple axial centerline to receive said staple distal end, wherein the improvements to said skin closure method comprising the steps of:
utilizing a base affixed to the proximal end of said staple and said base having a configuration that is adapted to provide a specific surface area for contact with said female member front portion, and wherein said base specific surface area is chosen so as to minimize the stresses that are applied to said tissue that is being joined,
utilizing a ratchet mechanism affixed to the outer surface of said staple and said ratchet mechanism having a configuration that is adapted to interact with and adjustably lock said staple distal end to said female member opening so that the axial length between said female member front portion and said staple base is adjustably set to a desired in-use, axial length which is commensurate with the thickness of the tissue which is to be joined,
utilizing an orientation, with respect to said staple axial centerline, for said staple base that is characterizible in terms of an angle, Ø, which is chosen so as to minimize the stresses that are applied to said tissue that is being joined, and utilizing a biodegradable material from which said staple is fabricated.

18. The improved skin closure method as recited in claim 17, wherein:
said ratchet mechanism configuration further adapted to detachably lock said staple distal end to said female member opening,
said staple outer surface having a configuration that is adapted to have a total surface area per unit volume of said staple that is set so as to help control the duration of time required for an implanted staple to biodegrade in a patient, and
said staple base and female member each having a configuration adapted to enable said device to be grasped in the hands of a caregiver and applied manually, without the aid of a fastening apparatus, to the edges of said tissue that is to be joined.

19. The improved skin closure method as recited in claim 18, wherein:
said biodegradable material is a combination of L-lactide and glycolide polymers, with the ratio of said polymers being in the range of 1/10 to 1/6,
said staple is fabricated by subjecting said staple to varying degrees of environmental factors in order to condition said staple such that said staple possesses the property that said staple biodegrades in a duration of between six to twenty days, and
said female member having a rear portion that includes a grasping member which has a configuration that is adapted to allow said grasping member to be grasped by a caregiver and used to help align said opening of said female member with said axial centerline of said staple.

20. The improved skin closure method as recited in claim 17, wherein the improvements to said skin closure method further comprising the step of:
utilizing a strip having an outer surface that includes a front portion with a cavity therein, and said cavity having a configuration adapted to accommodate said staple base and to detachably hold said staple in such a manner so as to aid in aligning the axial centerline of said staple with the opening of said female member during the adjustable locking of said staple distal end to said female member.
